(19) United States
(12) Patent Application Publication
(10) Pub. No.: US 2025/0352269 A1
(43) Pub. Date: Nov. 20, 2025

(54) POINT CLOUD NEURAL NETWORKS FOR LANDMARK ESTIMATION FOR ORTHOPEDIC SURGERY

(71) Applicant: Howmedica Osteonics Corp., Mahwah, NJ (US)

(72) Inventors: Yannick Morvan, Saint Renan (FR); Jérôme Ogor, Brest (FR); Jean Chaoui, Lyon (FR); Julien Ogor, Brest (FR); Thibaut Nico, Brest (FR)

(21) Appl. No.: 18/872,185

(22) PCT Filed: May 9, 2023
(86) PCT No.: PCT/US2023/021539

§ 371 Date: Dec. 5, 2024

Related U.S. Application Data
(60) Provisional application No. 63/350,752, filed on Jun. 9, 2022.

(51) Int. Cl. A61B 34/10; A61F 2/46; G06T 7/00; G06T 7/73; G16H 30/40

(52) U.S. Cl. CPC A61B 34/10; A61F 2/461; G06T 7/0012; G06T 7/73; G16H 30/40; A61B 2034/105; A61B 2034/107; A61F 2002/4633; G06T 2207/10028; G06T 2207/20081; G06T 2207/20084; G06T 2207/30008

(57) ABSTRACT

A computing system may be configured to obtain the first point cloud representing one or more bones of a patient, process the first point cloud using one or more point cloud neural networks to generate an output point cloud, the output point cloud including labels indicating locations of one or more landmarks on the one or more bones of the patient, and output the output point cloud.

This application claims priority to U.S. Provisional Patent Application 63/350,752, filed Jun. 9, 2022, the entire content of which is incorporated by reference.

### BACKGROUND

Surgical joint repair procedures involve repair and/or replacement of a damaged or diseased joint. A surgical joint repair procedure, such as joint arthroplasty as an example, may involve replacing the damaged joint with a prosthesis that is implanted into the patient's bone. For example, in a total shoulder replacement surgery, a surgeon may attach orthopedic prostheses to a scapula and a humerus of a patient. In an ankle replacement surgery, a surgeon may attach orthopedic prostheses to a tibia and a talus of a patient.

When planning an orthopedic surgery, it may be important for the surgeon to select appropriate tool alignment, such as a drilling axis, cutting plane, pin insertion axis, and so on. Selecting an inappropriate tool alignment may lead to improperly limited range of motion, an increased probability of failure of the orthopedic prosthesis, complications during surgery, and other adverse health outcomes. In addition, proper selection or design of a prosthetic that is appropriately sized and shaped and proper positioning of that prosthetic are important to ensure an optimal surgical outcome. A surgeon may analyze bone structure to assist with prosthetic selection, design and/or positioning, as well as surgical steps to prepare bone or tissue to receive or interact with a prosthetic.

### SUMMARY

This disclosure describes example techniques for automated estimation of landmarks (e.g., bony landmarks) on one or more bones of a patient to aid in orthopedic surgery planning. In particular, this disclosure describes techniques that include the application of a neural network to a 3D representation of one or more bones, where the neural network outputs a 3D representation including labels that identify the locations of the landmarks. In some examples, the 3D representation may be a point cloud and the neural network may be a neural network specifically configured to process an input point cloud (e.g., a point cloud neural network) and output an output point cloud that includes the labeled locations of landmarks. The output point cloud may be used in orthopedic surgery planning, including to visualize bones that are to be operated on, design and/or select surgical guides, design and/or select a prosthesis, design and/or select implant components that closely match the patient's anatomy, determine tool alignments, determine surgical cut locations, and for other surgical planning needs.

In one example, this disclosure describes a method for estimating landmarks on a morbid bone, the method comprising obtaining, by a computing system, a first point cloud representing one or more bones of a patient, processing, by the computing system, the first point cloud using one or more point cloud neural networks to generate an output point cloud, the output point cloud including labels indicating locations of one or more landmarks on the one or more bones of the patient, and outputting, by the computing system, the output point cloud.

In another example, this disclosure describes a computing system configured to estimate landmarks on a morbid bone, the computing system comprising a memory configured to store a first point cloud representing one or more bones of a patient. and one or more processors in communication with the memory, the one or more processors configured to obtain the first point cloud representing the one or more bones of the patient process the first point cloud using one or more point cloud neural networks to generate an output point cloud, the output point cloud including labels indicating locations of one or more landmarks on the one or more bones of the patient, and output the output point cloud.

In one example, this disclosure describes a non-transitory computer-readable storage medium having instructions stored thereon that, when executed, cause a computing system to obtain a first point cloud representing one or more bones of a patient, process the first point cloud using one or more point cloud neural networks to generate an output point cloud, the output point cloud including labels indicating locations of one or more landmarks on the one or more bones of the patient, and output the output point cloud.

The details of various examples of the disclosure are set forth in the accompanying drawings and the description below. Various features, objects, and advantages will be apparent from the description, drawings, and claims.

### BRIEF DESCRIPTION OF DRAWINGS

FIG. 1 is a block diagram illustrating an example system that may be used to implement the techniques of this disclosure.

FIG. 2 is a block diagram illustrating example components of a planning system, in accordance with one or more techniques of this disclosure.

FIG. 3 is a conceptual diagram illustrating an example point cloud neural network (PCNN), in accordance with one or more techniques of this disclosure.

FIG. 4 is a flowchart illustrating an example architecture of a T-Net model in accordance with one or more techniques of this disclosure.

FIG. 5 is a block diagram illustrating an example implementation of planning system according to one example of the disclosure.

FIG. 6 is a block diagram illustrating another example implementation of planning system according to another example of the disclosure.

FIG. 7 is a block diagram illustrating another example implementation of planning system according to another example of the disclosure.

FIG. 8 is a conceptual diagram illustrating an example technique for training a neural network in accordance with one or more techniques of this disclosure.

FIG. 9 is a conceptual diagram illustrating an example output point cloud with labeled landmarks in accordance with one or more techniques of this disclosure.

FIG. 10 is a flowchart illustrating an example process for estimating landmarks in accordance with one or more techniques of this disclosure.

### DETAILED DESCRIPTION

When planning an orthopedic surgery, it may be important for the surgeon to design and/or select surgical guides, design and/or select a prosthesis, design and/or select implant components that closely match the patient's anatomy, determine tool alignments, determine surgical cut locations, and determine other surgical planning needs. This may be difficult in many situations, as the bone to be operated on may be a morbid bone having one or more deformities due to disease. A “morbid” bone may also be referred to as a pathologic bone. In some examples, a bone may have been operated on previously and may already have a prosthesis implanted. In addition, in some examples, a bone may have been operated on previously and may already have a prosthesis implanted. Such deformities may include additive deformities, such as ossification, where additional bone material is present in relation to a healthy, pre-morbid bone. Other deformities may be subtractive, such as bone erosion, where bone material from a pre-morbid bone is no longer present on the patient's current, morbid mode.

This disclosure describes techniques that may address one or more challenges associated with planning orthopedic surgeries. For instance, in accordance with one or more techniques of this disclosure, a computing system may obtain a first point cloud representing one or more bones of a patient. The computing system may process the first point cloud using one or more point cloud neural networks to generate an output point cloud, the output point cloud including labels indicating locations of one or more landmarks on the one or more bones of the patient. In some examples, the output of the computing system may be only the labels and their associated locations. For example, the output of the computing system may be a list of labels. Each label in the list is the part of the bone/landmark the computing system determines that the ith point (e.g., a particular point) of the input point cloud belongs to. The computing system may output the output point cloud, for example, for future review study during surgical planning. In some examples, the output point cloud, including labels of the landmarks, may be visualized using one or more of a mixed reality (MR) visualization device, a virtual reality (VR) visualization device, a holographic projector, or an extended reality (XR) visualization device.

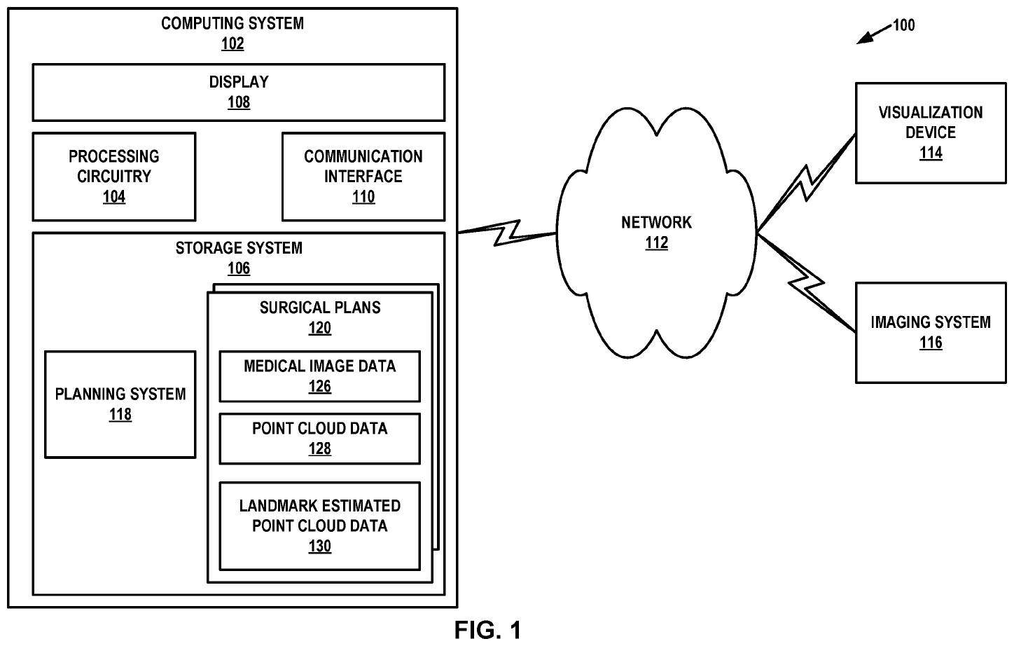

FIG. 1 is a block diagram illustrating an example system 100 that may be used to implement the techniques of this disclosure. FIG. 1 illustrates computing system 102, which is an example of one or more computing devices that are configured to perform one or more example techniques described in this disclosure. Computing system 102 may include various types of computing devices, such as server computers, personal computers, smartphones, laptop computers, and other types of computing devices. In some examples, computing system 102 includes multiple computing devices that communicate with each other. In other examples, computing system 102 includes only a single computing device. Computing system 102 includes processing circuitry 104, storage system 106, a display 108, and a communication interface 110. Display 108 is optional, such as in examples where computing system 102 is a server computer.

Examples of processing circuitry 104 include one or more microprocessors, digital signal processors (DSPs), application specific integrated circuits (ASICs), field programmable gate arrays (FPGAs), discrete logic, software, hardware, firmware or any combinations thereof. In general, processing circuitry 104 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits. In some examples, processing circuitry 104 is dispersed among a plurality of computing devices in computing system 102 and visualization device 114. In some examples, processing circuitry 104 is contained within a single computing device of computing system 102.

Processing circuitry 104 may include arithmetic logic units (ALUs), elementary function units (EFUs), digital circuits, analog circuits, and/or programmable cores, formed from programmable circuits. In examples where the operations of processing circuitry 104 are performed using software executed by the programmable circuits, storage system 106 may store the object code of the software that processing circuitry 104 receives and executes, or another memory within processing circuitry 104 (not shown) may store such instructions. Examples of the software include software designed for surgical planning.

Storage system 106 may be formed by any of a variety of memory devices, such as dynamic random access memory (DRAM), including synchronous DRAM (SDRAM), magnetoresistive RAM (MRAM), resistive RAM (RRAM), or other types of memory devices. Examples of display 108 include a liquid crystal display (LCD), a plasma display, an organic light emitting diode (OLED) display, or another type of display device. In some examples, storage system 106 may include multiple separate memory devices, such as multiple disk drives, memory modules, etc., that may be dispersed among multiple computing devices or contained within the same computing device.

Communication interface 110 allows computing system 102 to communicate with other devices via network 112. For example, computing system 102 may output medical images, images of segmentation masks, and other information for display. Communication interface 110 may include hardware circuitry that enables computing system 102 to communicate (e.g., wirelessly or using wires) to other computing systems and devices, such as a visualization device 114 and an imaging system 116. Network 112 may include various types of communication networks including one or more wide-area networks, such as the Internet, local area networks, and so on. In some examples, network 112 may include wired and/or wireless communication links.

Visualization device 114 may utilize various visualization techniques to display image content to a surgeon. In some examples, visualization device 114 is a computer monitor or display screen. In some examples, visualization device 114 may be a mixed reality (MR) visualization device, virtual reality (VR) visualization device, holographic projector, or other device for presenting extended reality (XR) visualizations. For instance, in some examples, visualization device 114 may be a Microsoft HOLOLENS™ headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses. In some examples, there may be multiple visualization devices for multiple users.

Visualization device 114 may utilize visualization tools that are available to utilize patient image data to generate three-dimensional models of bone contours, bony landmarks, segmentation masks, or other data to facilitate preoperative planning. These tools may allow surgeons to design and/or select surgical guides and implant components that closely match the patient's anatomy. These tools can improve surgical outcomes by customizing a surgical plan for each patient. An example of such a visualization tool is the BLUEPRINT™ system available from Stryker Corp. The surgeon can use the BLUEPRINT™ system to select, design or modify appropriate implant components, determine how best to position and orient the implant components and how to shape the surface of the bone to receive the components, and design, select or modify surgical guide tool(s) or instruments to carry out the surgical plan. The information generated by the BLUEPRINT™ system may be compiled in a preoperative surgical plan for the patient that is stored in a database at an appropriate location, such as storage system 106, where the preoperative surgical plan can be accessed by the surgeon or other care provider, including before and during the actual surgery.

Imaging system 116 may comprise one or more devices configured to generate medical image data. For example, imaging system 116 may include a device for generating CT images. In some examples, imaging system 116 may include a device for generating MRI images. Furthermore, in some examples, imaging system 116 may include one or more computing devices configured to process data from imaging devices in order to generate medical image data. For example, the medical image data may include a 3D image of one or more bones of a patient. In this example, imaging system 116 may include one or more computing devices configured to generate the 3D image based on CT images or MRI images.

Computing system 102 may obtain a point cloud representing one or more bones of a patient. The point cloud may be generated based on the medical image data generated by imaging system 116. In some examples, imaging system 116 may include one or more computing devices configured to generate the point cloud. Imaging system 116 or computing system 102 may generate the point cloud by identifying the surfaces of the one or more bones in images and sampling points on the identified surfaces. Each point in the point cloud may correspond to a set of 3D coordinates of a point on a surface of a bone of the patient. In other examples, computing system 102 may include one or more computing devices configured to generate the medical image data based on data from devices in imaging system 116. The point clouds may represent any number of bones. For example, for ankle-related surgeries, it may be useful to visualize point clouds of tibia and fibula bones. For shoulder-related surgeries, it may be useful to visualize a point cloud depicting the humerus. However, the techniques of this disclosure are not limited to any particular bone or set of bones.

In some examples, the input point clouds may be so-called “enriched point clouds of size n×6, where n is the number points. The “6” dimension indicates that each point of the point cloud includes an (x, y, z) location as well as normal coordinates (nx, ny, nz). In some examples, the point cloud may further include additional information, such as curvature information, label information, or other information on top of the point cloud coordinates. In some examples, the additional information may be generated by the techniques of this disclosure.

Storage system 106 of computing system 102 may store instructions that, when executed by processing circuitry 104, cause computing system 102 to perform various activities. For instance, in the example of FIG. 1, storage system 106 may store instructions that, when executed by processing circuitry 104, cause computing system 102 to perform activities associated with a planning system 118. For ease of explanation, rather than discussing computing system 102 performing activities when processing circuitry 104 executes instructions, this disclosure may simply refer to planning system 118 or components thereof as performing the activities or may directly describe computing system 102 as performing the activities.

In the example of FIG. 1, storage system 106 stores surgical plans 120. Surgical plans 120 may correspond to individual patients. A surgical plan corresponding to a patient may include data associated with a planned or completed orthopedic surgery on the corresponding patient. A surgical plan corresponding to a patient may include medical image data 126 for the patient, point cloud data 128, and landmark estimated point cloud data 130 for the patient. Medical image data 126 may include computed tomography (CT) images of bones of the patient or 3D images of bones of the patient based on CT images. In this disclosure, the term “bone” may refer to a whole bone or a bone fragment. In some examples, medical image data 126 may include magnetic resonance imaging (MRI) images of one or more bones of the patient or 3D images based on MRI images of the one or more bones of the patient. In some examples, medical image data 126 may include ultrasound images of one or more bones of the patient. Point cloud data 128 may include point clouds representing bones of the patient. landmark estimated point cloud data 130 may include data representing one or more point clouds of a patient's morbid bone that include labels of where one or more bony landmarks are estimated to have been located in a pre-morbid version of the patient's bone. Example techniques that describe how computing system 102 generates landmark estimated point cloud data 130 will be described in more detail below.

Planning system 118 may be configured to assist a surgeon with planning an orthopedic surgery that may include the design and/or selection of surgical guides, the design and/or selection of a prosthesis, the design and/or selection of implant components that closely match the patient's anatomy, a determination of tool alignments, a determination of surgical cut locations, and determine other surgical planning needs. In accordance with one or more techniques of this disclosure, planning system 118 may apply a point cloud neural network (PCNN) to generate an output point cloud based on an input point cloud. Point cloud data 128 may include the input point cloud and/or the output point cloud. The input point cloud represents one or more bones of the patient. In some examples, the output point cloud includes labels indicating locations of one or more landmarks on one or more bones of a patient. The output point cloud may be viewed on visualization device 114. In some examples, the output of the PCNN may be only the labels and their associated locations. For example, the output of the PCNN may be a list of labels. Each label in the list is the part of the bone/landmark the PCNN determines that the ith point (e.g., a particular point) of the input point cloud belongs to.

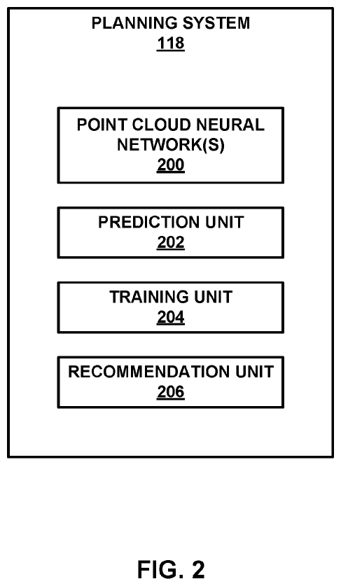

FIG. 2 is a block diagram illustrating example components of planning system 118, in accordance with one or more techniques of this disclosure. In the example of FIG. 2, the components of planning system 118 include a PCNN 200, a prediction unit 202, a training unit 204, and a recommendation unit 206. In other examples, planning system 118 may be implemented using more, fewer, or different components. For instance, training unit 204 may be omitted in instances where PCNN 200 has already been trained. In some examples, one or more of the components of planning system 118 are implemented as software modules. In other examples, as will be described below, planning system may include multiple PCNNs. Moreover, the components of FIG. 2 are provided as examples and planning system 118 may be implemented in other ways.

Prediction unit 202 may apply PCNN 200 to generate an output point cloud based on an input point cloud. The input point cloud represents one or more bones of a patient. In some examples, the output point cloud includes labels indicating locations of one or more landmarks on the one or more bones of the patient. That is, the output point cloud includes a plurality of points that generally represent the shape of a pre-morbid bone, including points representing landmarks (e.g., bony landmarks) in the pre-morbid bone. In this context, a pre-morbid bone may be the estimated shape of the patient's bone before disease and/or deterioration or before a previous implantation of a prosthesis. The points representing the landmarks may further include labels that indicate particular landmarks. For ankle-related surgeries, the bones represented in the input and output point clouds may include the tibia and the fibula. Example landmarks that may be labeled in the output point cloud may include one or more of an anterior crest, a medial malleolus, a soleal line, a fibular facet, an articulation site for a head of the fibula, a fibular notch, an articulation site for a distal fibula, a point of ligamentous attachment, a proximal head of the fibula, a lateral malleolus, a facet on a distal end of the fibula, a medial condyle, a lateral condyle, a tibial plateau, a tibial tuberosity, a distal tibia plafond landmark, a proximal tibia knee center, a tibia canal center, medial tibia gutter points, and/or a site for articulation with a talus bone.

In some examples, the labels of the landmarks on the output point cloud may be metadata associated with a point or group of points. The metadata may indicate the particular landmark that the point or group of points is associated with. The metadata may also indicate if a particular landmark corresponds to a healthy portion of the bone or a deformed portion of the bone. Such metadata may help in classifying which part of the bone can be used as input for the prediction of the pre-morbid shape of the bone. In some examples, the metadata itself may be visible when viewing the output point cloud. In other examples, the labels of locations of landmarks may take the form of visibly changing the appearance of points in the output point cloud associated with a particular landmark. For example, points labeled as being associated with a particular landmark may be displayed with different colors, patterns, shading, or other visible markers that designate points belonging to a particular landmark to other points in the output point cloud. The techniques of this disclosure are not limited to any particular technique whereby points in an output point cloud are labeled as belonging to a particular landmark.

Prediction unit 202 may obtain the input point cloud in one of a variety of ways. For example, prediction unit 202 may generate the input point cloud based on medical image data. The medical image data for the patient may include a plurality of input images (e.g., CT images or MRI images, etc.). In this example, each of the input images may have a width dimension and a height dimension, and each of the input images may correspond to a different depth-dimension layer in a plurality of depth-dimension layers. In other words, the plurality of input images may be conceptualized as a stack of 2D images, where the positions of individual 2D images in the stack in the depth dimension. As part of generating the point cloud, prediction unit 202 may perform an edge detection algorithm (e.g., Canny edge detection, Phase Stretch Transform (PST), etc.) on the 2D images (or a 3D image based on the 2D images). Prediction unit 202 may select points on the detected edges as points in the point cloud. In other examples, prediction unit 202 may obtain the input point cloud from one or devices outside of computing system 102.

As indicated above, the output point cloud may, in some examples, include points indicating locations of estimated landmarks in a pre-morbid version of a patient's bone. In some such examples, the output point cloud is limited to points indicating the landmarks. In other words, the output point cloud does not include points representing other bone or other tissue of the patient. In some examples, the output point cloud includes points indicating the landmarks and points representing other objects, such as other regions of the bones or tissues of the patient, estimated contours of a pre-morbid bone, estimated surgical cut locations, and/or estimated tool alignments.

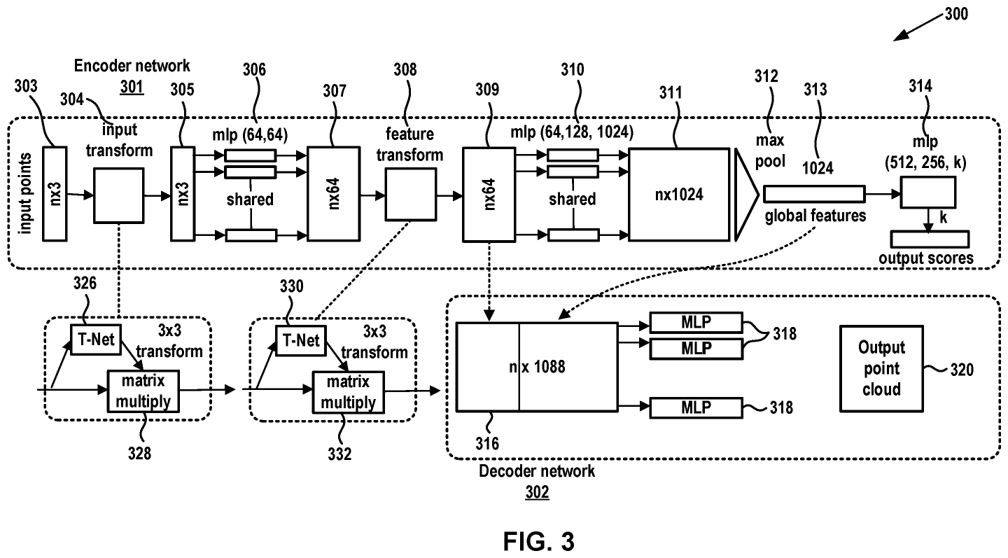

In one example, PCNN 200 is implemented using a point cloud learning model-based architecture. A point cloud learning model-based architecture (e.g., a point cloud learning model) is a neural network-based architecture that receives one or more point clouds as input and generates one or more point clouds as output. Example point cloud learning models include PointNet, PointTransformer, and so on. An example point cloud learning model-based architecture based on PointNet is described below with respect to FIG. 3.

While the example of FIG. 2 shows a PCNN, other types of neural networks may be in conjunction with the techniques of this disclosure. In some examples, PCNN 200 may be one or more artificial neural networks (ANNs), including deep neural networks (DNNs) and/or convolutional neural networks (CNNs). In general, neural networks have shown great promise as classification tools. PCNN 200 may include an input layer, an output layer, and one or more hidden layers between the input layer and the output layer. PCNN 200 may also include one or more other types of layers, such as pooling layers.

Each layer may include a set of artificial neurons, which are frequently referred to simply as “neurons.” Each neuron in the input layer receives an input value from an input vector. Outputs of the neurons in the input layer are provided as inputs to a next layer in the network. Each neuron of a layer after the input layer may apply a propagation function to the output of one or more neurons of the previous layer to generate an input value to the neuron. The neuron may then apply an activation function to the input to compute an activation value. The neuron may then apply an output function to the activation value to generate an output value for the neuron. An output vector of the network includes the output values of the output layer of the network.

Each output layer neuron in the plurality of output layer neurons corresponds to a different output element in a plurality of output elements. Each output element in the plurality of output elements corresponds to a different classification. In the example of this disclosure, the classifications may be points identified as being a location of a bony landmark in a pre-morbid version of a patient's bone. In this example, a computing system, such as computing system 102 may receive a plurality of training datasets that include annotated 3D representations (e.g., point clouds or 3D images) of a patient's bone(s). The annotated representations may include points that are manually identified as being the location of a pre-morbid landmark. The annotated representation may also include points with labels indicating healthy (e.g., pre-morbid) and not healthy (e.g., morbid/deformed). Each respective training dataset corresponds to a different training data patient in a plurality of training data patients and comprises a respective training input vector and a respective target output vector.

For each respective training dataset, the training input vector of the respective training dataset comprises a value for each element of the plurality of input elements. For each respective training dataset, the target output vector of the respective training dataset comprises a value for each element of the plurality of output elements. In this example, computing system 102 may use the plurality of training datasets to PCNN 200. As will be explained in more detail below, training PCNN 200 may include determining parameters of PCNN 200 by minimizing a loss function. The parameters of PCNN 200 may include weights applied to the output layers of the neural network and/or output functions for the layers of the neural network.

In one example, the 3D representations of bones processed by PCNN 200 are point clouds. In one example, to process point cloud data, PCNN 200 may be a neural network with a plurality of layers configured to classify (e.g., segment) three-dimensional point cloud input data. As mentioned above, one example of such a neural network is PointNet. PointNet, or other similarly configured convolutional neural networks, may have a fully connected network structure using one or more pooling layers (e.g., global or local pooling layers). Convolutional neural networks convolve the input of a layer and pass the result to the next layer. A network structure has fully connected layers if every neuron in one layer is connected to every neuron in another layer. A network with fully connected layers may also be called a multi-layer perceptron neural network (MLP).

In some examples, a pooling layer reduces the dimensions of data by combining the outputs of neurons at one layer into a single neuron in the next layer. Local pooling combines small data clusters. Global pooling involves all the neurons of the network. Two common types of pooling include max pooling and average pooling. As one example, PointNet uses max pooling. Max pooling uses the maximum value of each local cluster of neurons in the network.

In some examples, each neuron in PCNN 200 computes an output value by applying a specific function to the input values received from the previous layer. The function that is applied to the input values is determined by a vector of weights and bias values. The weights and bias values for PCNN 200 may be included in a set of parameters. As will be explained below, training PCNN 200 may include iteratively adjusting these weights and bias values. The vector of weights and a bias value is sometimes called a filter and may represent particular features to be segmented. In this example, the particular features to be segmented may include the estimated locations of landmarks (e.g., bony landmarks) in a patient's pre-morbid bone.

PCNN 200 is effectively trained to learn a set of optimization functions that determine estimated locations of landmarks in a patient's pre-morbid bone. PCNN 200 may encode an output that indicates a reason for the determination of estimated locations of landmarks in a patient's pre-morbid bone. In one example, fully connected layers of PCNN 200 aggregate learned optimal values into a descriptor that is used to predict per point labels (i.e., labelling points as being part of a landmark). In one example, PCNN 200 is configured as a classification network that takes n points as input, applies input and feature transformations, and then aggregates point features by max pooling.

In another example of the disclosure, rather than processing point clouds, planning system 118 may be configured to process 3D binary images. In this example, to process a 3D image, the neural network employed by planning system 118 may be a neural network with a plurality of layers configured to classify (e.g., segment) three-dimensional image (e.g., voxels) input data. In this example, the neural network may be a convolutional neural network, such as Vnet or Unet. Such a neural network may be trained and operated similarly to what is described above, adjusting for a different input structure (e.g., images vs. point clouds).

Planning system 118 may include different sets of PCNNs for different surgery types. The set of PCNNs for a surgery type may include one or more PCNNs corresponding to different instances where the surgeon desires to perform one or more pre-surgery planning processes for a particular surgery type. For example, the set of PCNNs for a total ankle replacement surgery may include a first PCNN that generates an output point cloud that includes landmarks on a distal end of a tibia. In this example, a second PCNN of the set of PCNNs for the total ankle replacement surgery may generate an output point cloud that includes landmarks on a distal end of a fibula.

Training unit 204 may train PCNN 200. For instance, training unit 204 may generate a plurality of training datasets. Each of the training datasets may correspond to a different historic patient in a plurality of historic patients. The historic patients may include patients for whom surgical plans have been developed. For instance, surgical plans 120 (FIG. 1) may include surgical plans for the historic patients. In some examples, the surgical plans may be limited to those developed by expert surgeons, e.g., to ensure high quality training data. In some examples, the historic patients may be selected for relevance. The surgical plans may include data indicating estimated locations of landmarks on a pre-morbid bone.

The training dataset for a historic patient may include training input data and expected output data. The training input data may include a point cloud representing one or more bones of the patient. In examples where PCNN 200 generates output point clouds indicating estimated locations of landmarks on a pre-morbid bone, the expected output data comprises a point cloud that includes points indicating locations of landmarks on a pre-morbid bone on historic patients. In some examples, training unit 204 may generate the training input data based on medical image data stored in surgical plans of historic patients. In some examples, training unit 204 may generate the expected outpoint data based on the location of landmarks in the surgical plans of historic patients.

Training unit 204 may train PCNN 200 based on the training datasets. Because training unit 204 generates the training datasets based on how real surgeons actually planned and/or executed surgeries in historic patients, a surgeon who ultimately uses and/or visualizes the output point clouds generated by planning system 118 may have confidence that the labeled landmarks are based on how other real surgeons have identified landmarks for real historic patients.

In some examples, as part of training PCNN 200, training unit 204 may perform a forward pass on the PCNN 200 using the input point cloud of a training dataset as input to PCNN 200. Training unit 204 may then perform a process that compares the resulting output point cloud generated by PCNN 200 to the corresponding expected output point cloud. In other words, training unit 204 may use a loss function to calculate a loss value based on the output point cloud generated by PCNN 200 and the corresponding expected output point cloud. In some examples, the loss function is targeted at minimizing a difference between the output point cloud generated by PCNN 200 and the corresponding expected output point cloud. Examples of the loss function may include a Chamfer Distance (CD) and the Earth Mover's Distance (EMD). The CD may be given by the average of a first average and a second average. The first average is an average of distances between each point in the output point cloud generated by PCNN 200 and its closest point in the expected output point cloud. The second average is an average of distances between each point in the expected output point cloud and its closest point in the output point cloud generated by PCNN 200. The CD may be defined as:

$${ℒ}_{CD} ( {S}_{1} , {S}_{2} ) = \frac{1}{2} ( \frac{1}{\vert {S}_{1} \vert} {\sum }_{x ∈ {S}_{1}} {min}_{y ∈ {S}_{2}} ‖ x - y ‖ + \frac{1}{\vert {S}_{2} \vert} {\sum }_{y ∈ {S}_{2}} {min}_{x ∈ {S}_{1}} ‖ x - y ‖ )$$

In the equation above, S₁ is the output point cloud generated by PCNN 200, S₂ is the expected output point cloud, | . . . | is an element indicating number of elements, and ∥ . . . ∥ indicates absolute value.

Training unit 204 may then perform a backpropagation process based on the loss value to adjust parameters of PCNN 200 (e.g., weights of neurons of PCNN 200). In some examples, training unit 204 may determine an average loss value based on loss values calculated from output point clouds generated by performing multiple forward passes through PCNN 200 using different input point clouds of the training data. In such examples, training unit 204 may perform the backpropagation process using the average loss value to adjust the parameters of PCNN 200. Training unit 204 may repeat this process during multiple training epochs.

During use of PCNN 200 (e.g., after training of PCNN 200), prediction unit 202 of planning system 118 may apply PCNN 200 to generate an output point cloud for a patient based on an input point cloud representing one or more bones of the patient. In some examples, recommendation unit 206 may determine one or more of a cut location, implant design, implant size, tool alignment, prostheses design, and/or prostheses size based on the location of one or more landmarks labeled in the output point cloud.

In some examples, recommendation unit 206 may output for display one or more images (e.g., one or more 2D or 3D images) or models showing the output point cloud with identified landmarks. For example, recommendation unit 206 may output for display an image showing a 3D representation of the output point cloud indicating one or more visual indications of bony landmarks identified by PCNN 200. In some such examples, the output point cloud generated by PCNN 200 and the input point cloud (which represents one or more bones of the patient) are in the same coordinate system. Accordingly, recommendation unit 206 may overlay the input point cloud and the output point cloud to better illustrate where the landmarks may have been located on a pre-morbid version of the patient's bone.

In some examples, recommendation unit 206 may generate, based on the output point cloud, a MR visualization indicating estimated landmark locations. In examples where visualization device 114 (FIG. 1) is an MR visualization device, visualization device 114 may display the MR visualization. In some examples, visualization device 114 may display the MR visualization during a planning phase of a surgery. In such examples, recommendation unit 206 may generate the MR visualization as a 3D image in space. Recommendation unit 206 may generate the 3D image in the same as described above for generating the 3D image.

In some examples, the MR visualization is an intra-operative MR visualization. In other words, visualization device 114 may display the MR visualization during surgery. In some examples, visualization device 114 may perform a registration process that registers the MR visualization with the physical bones of the patient. Accordingly, in such examples, a surgeon wearing visualization device 114 may be able to see estimated landmarks relative to a bone of the patient.

In addition to determining and/or displaying estimated landmarks relative to a bone of the patient, computing system 102 may also be configured to determine one or more axes, planes, and lines that describes aspects of human anatomy based on the determined landmarks, including the locations of the landmarks. As examples, based on the estimated landmarks, computing system 102 may determine one or more of a tibia mechanical axis, a tibia anatomical axis, a tibia axial plane, a tibia medial gutter line, a fibula lateral gutter line, a tibial mortise AP (anteroposterior) axis, and/or a tibial mortise ML (mediolateral) axis. A tibia mechanical axis is a line passing through the distal tibia plafond landmark and the proximal tibia knee center. The landmarks estimated by computing system 102 may include the distal tibia plafond landmark and the proximal tibia knee center. A tibia anatomical axis is a line passing through the distal tibial plafond landmark and the tibia canal center landmark. The landmarks estimated by computing system 102 may further include the tibia canal center landmark. A tibia axial plane is a plane perpendicular to the mechanical axis and passing through the distal tibia plafond landmark. The tibia medial gutter line is a best-fit line through the medial tibia gutter points projected onto the tibia axial plane. The landmarks estimated by computing system 102 may include the medial tibia gutter points. The tibial mortise AP axis is a bisection between the medial and lateral tibial gutter lines in the tibia axial plane. The landmarks estimated by computing system 102 may include the medial and lateral tibial gutter points. The tibial mortise ML axis is the line perpendicular to the AP axis on the axial plan passing through the tibia plafond landmark. Recommendation unit 206 may also be configured to generate, based on the output point cloud, a MR visualization indicating estimated landmark locations as well as one or more of the determined axes, planes, and lines described above.

FIG. 3 is a conceptual diagram illustrating an example point cloud learning model 300 in accordance with one or more techniques of this disclosure. Point cloud learning model 300 may receive an input point cloud. The input point cloud is a collection of points. The points in the collection of points are not necessarily arranged in any specific order. Thus, the input point cloud may have an unstructured representation.

In the example of FIG. 3, point cloud learning model 300 includes an encoder network 301 and a decoder network 302. Encoder network 301 receives an array 303 of n points. The points in array 303 may be the input point cloud of point cloud learning model 300. In the example of FIG. 3, each of the points in array 303 has a dimensionality of 3. For instance, in a Cartesian coordinate system, each of the points may have an x coordinate, a y coordinate, and a z coordinate.

Encoder network 301 may apply an input transform 304 to the points in array 303 to generate an array 305. Encoder network 301 may then use a first shared multi-layer perceptron (MLP) 306 to map each of the n points in array 305 from three dimensions to a larger number of dimensions a (e.g., a=64 in the example of FIG. 3), thereby generating an array 307 of n×a (e.g., n×64 values). For ease of explanation, the following description of FIG. 3 assumes that a is equal to 64 but in other examples other values of a may be used. Encoder network 301 may then apply a feature transform 308 to the values in array 307 to generate an array 309 of n×64 values. For each of the n points in array 309, encoder network 301 uses a second shared MLP 310 to map the n points from a dimension to b dimensions (e.g., b=1024 in the example of FIG. 3), thereby generating an array 311 of n×b (e.g., n×1024 values). For ease of explanation, the following description of FIG. 3 assumes that b is equal to 1024 but in other examples other values of b may be used. Encoder network 301 applies a max pooling layer 312 to generate a global feature vector 313. In the example of FIG. 3, each of points n in global feature vector 313 has 1024 dimensions.

Thus, as part of applying an PCNN 200, computing system 102 may apply an input transform (e.g., input transform 304) to a first array (e.g., array 303) that comprises the point cloud to generate a second array (e.g., array 305), wherein the input transform is implemented using a first T-Net model (e.g., T-Net Model 326), apply a first MLP (e.g., MLP 306) to the second array to generate a third array (e.g., array 307), apply a feature transform (e.g., feature transform 308) to the third array to generate a fourth array (e.g., array 309), wherein the input transform is implemented using a second T-Net model (e.g., T-Net model 330), apply a second MLP (e.g., MLP 910) to the fourth array to generate a fifth array (e.g., array 311); and apply a max pooling layer (e.g., max pooling layer 312) to the fifth array to generate the global feature vector (e.g., global feature vector 313).

A fully-connected network 314 may map global feature vector 313 to k output classification scores. The value k is an integer indicating a number of classes. Each of the output classification scores corresponds to a different class. An output classification score corresponding to a class may indicate a level of confidence that the input point cloud as a whole corresponds to the class. Fully-connected network 314 includes a neural network having two or more layers of neurons in which each neuron in a layer is connected to each neuron in a subsequent layer. In the example of FIG. 3, fully-connected network 314 includes an input layer having 512 neurons, a middle layer having 256 neurons, and an output layer having k neurons. In some examples, fully-connected network 314 may be omitted from encoder network 301.

Input 916 to decoder network 302 may be formed by concatenating the n 64-dimensional points of array 309 with global feature vector 313. In other words, for each point of the n points in array 309, the corresponding 64 dimensions of the point are concatenated with the 1024 features in global feature vector 313.

Decoder network 302 may sample N points in a unit square in 2-dimensions. Thus, decoder network 302 may randomly determine N points having x-coordinates in a range of [0,1] and y-coordinates in the range of [0,1]. Decoder network 302 may concatenate the sampled points with global feature vector 313 to obtain a combined vector 316. Decoder network 302 may apply K MLPs 318 (where K is an integer greater than or equal to 1) to the combined vector to generate a single point in the output point cloud. Each of the K MLPs 318 may generate points in a different patch (e.g., area) of the output point cloud. Each of the MLPs 318 may reduce the number of features from 1026 to 3. The 3 features may correspond to the 3 coordinates of a point of the output point cloud. For instance, for each sampled point n in N, the MLPs 318 may reduce the features from 1026 to 512 to 256 to 128 to 64 to 3. Thus, a K×N×3 vector may contain an output point cloud 920. In some examples, as part of training the MLPs of decoder network 302, decoder network 302 may calculate a chamfer loss of an output point cloud relative to a ground-truth point cloud. Decoder network 302 may use the chamfer loss in a backpropagation process to adjust parameters of the MLPs. In this way, planning system 118 may apply the decoder (e.g., decoder network 302) to generate the premorbid bone model based on the global feature vector.

Input transform 304 and feature transform 308 in encoder network 301 may provide transformation invariance. In other words, point cloud learning model 300 may be able to generate output point clouds (e.g., output bone models with labeled landmarks) in the same way, regardless of how the input point cloud (e.g., input bone model) is rotated, scaled, or translated. The fact that point cloud learning model 300 provides transform invariance may be advantageous because it may reduce the susceptibility of generator ML model 128 to errors based on positioning/scaling in morbid bone models. As shown in the example of FIG. 3, input transform 304 may be implemented using a T-Net Model 326 and a matrix multiplication operation 328. T-Net Model 326 generates a 3×3 transform matrix based on array 303. Matrix multiplication operation 328 multiplies array 303 by the 3×3 transform matrix. Similarly, feature transform 308 may be implemented using a T-Net model 330 and a matrix multiplication operation 332. T-Net model 330 may generate a 64×64 transform matrix based on array 307. Matrix multiplication operation 328 multiplies array 307 by the 64×64 transform matrix.

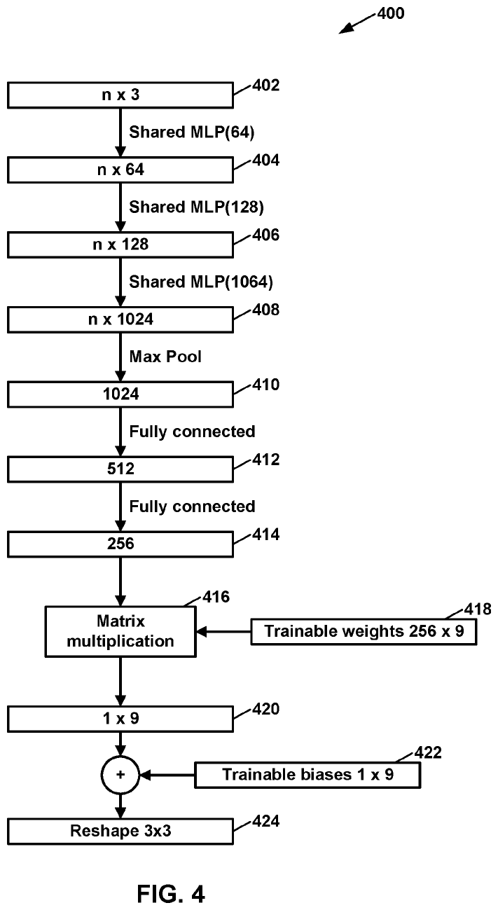

FIG. 4 is a block diagram illustrating an example architecture of a T-Net model 400 in accordance with one or more techniques of this disclosure. T-Net model 400 may implement T-Net Model 326 used in the input transform 304. In the example of FIG. 4, T-Net model 400 receives an array 402 as input. Array 402 includes n points. Each of the points has a dimensionality of 3. A first shared MLP maps each of the n points in array 402 from 3 dimensions to 64 dimensions, thereby generating an array 404. A second shared MLP maps each of the n points in array 404 from 64 dimensions to 128 dimensions, thereby generating an array 406. A third shared MLP maps each of the n points in array 406 from 128 dimensions to 1024 dimensions, thereby generating an array 408. T-Net model 400 then applies a max pooling operation to array 408, resulting in an array 810 of 1024 values. A first fully-connected neural network maps array 410 to an array 812 of 512 values. A second fully-connected neural network maps array 412 to an array 414 of 256 values. T-Net model 400 applies a matrix multiplication operation 416 to a matrix of trainable weights 418. The matrix of trainable weights 418 has dimensions of 256×9. Thus, multiplying array 414 by the matrix of trainable weights 418 results in an array 820 of size 1×9. T-Net model 400 may then add trainable biases 422 to the values in array 420. A reshaping operation 424 may remap the values resulting from adding trainable biases 422 into a 3×3 transform matrix. In other examples, the sizes of the matrixes and arrays may be different.

T-Net model 330 (FIG. 3) may be implemented in a similar way as T-Net model 400 in order to perform feature transform 308. However, in this example, the matrix of trainable weights 418 is 256×4096 and the trainable biases 422 has size 1×4096 bias values instead of 9. Thus, the T-Net model for performing feature transform 308 may generate a transform matrix of size 64×64. In other examples, the sizes of the matrixes and arrays may be different.

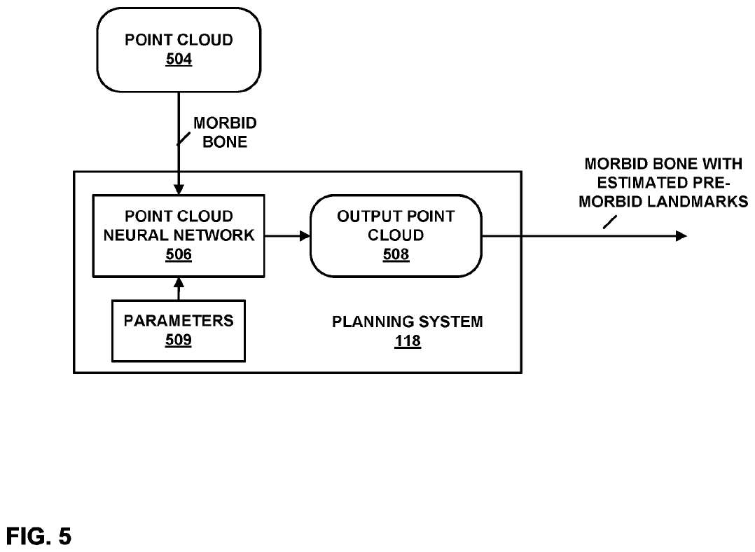

FIG. 5 is a block diagram illustrating an example implementation of planning system 118 according to one example of the disclosure. In the example of FIG. 5, planning system 118 obtains and receives point cloud 504 as input. Point cloud 504 is a point cloud representing one or more morbid bones of a patient. That is, point cloud 504 represents the current status of the bones of a patient on which surgery is being planned. Planning system 118 then processes point cloud 504 using point cloud neural network 506.

Point cloud neural network 506 is one example of PCNN(s) 200 of FIG. 2. In the example of FIG. 5, point cloud neural network 506 is trained to process a point cloud representing one or more morbid bones of a patient and to output a point cloud of the morbid bone with estimated pre-morbid landmarks. That is, output point cloud 508 includes labeled points that indicate the estimated location of one or more bony landmarks of the patient's bones prior to disease or deterioration (e.g., a pre-morbid bone). By estimating the location of landmarks in a pre-morbid bone, a surgeon may better understand the shape, size, and design of a prosthesis needed to best recreate pre-morbid conditions in a patient's joint. In addition, the location of landmarks in a pre-morbid bone may also aid the determination of cut locations, tool alignments, or other pre-surgery plans.

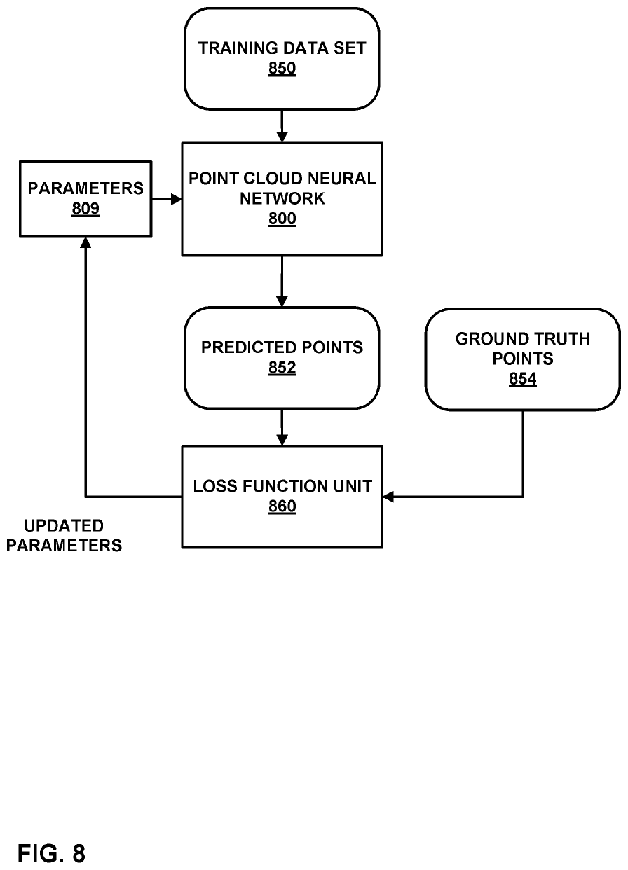

Point cloud neural network 506 may process point cloud 504 according to parameters 509. As described above, parameters 509 may include biases and weights for one or more layers of point cloud neural network 506. Point cloud neural network 506 may be pre-trained or trained by planning system 118. A training data set for point cloud neural network 506 may include a plurality of point clouds of morbid bones that include annotations indicating the expected location of a landmark to be estimated by point cloud neural network. For example, one or more surgeons or other experts may annotate point clouds of morbid bones to indicate the expected locations of certain bony landmarks. Such training data sets may then be processed by point cloud neural network 506 and the predicted landmark points in the output point cloud produced by point cloud neural network 506 may be compared to the annotated locations (e.g., the ground truth points) and a loss function may be calculated. Based on the output of the loss function, the weights and bias values in parameters 509 may be updated and the training data set may be processed again to further refine the parameters. This process is iteratively repeated until a desired value for the loss function is achieved. In general, the smaller the value of the loss function, the more accurate the output of the neural network. An example of training a neural network is described below with reference to FIG. 8.

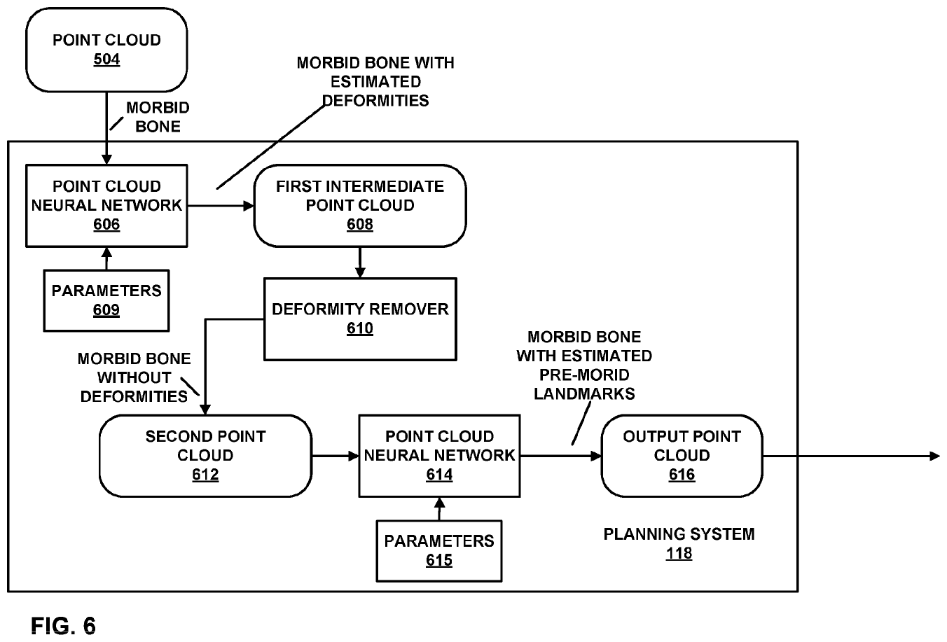

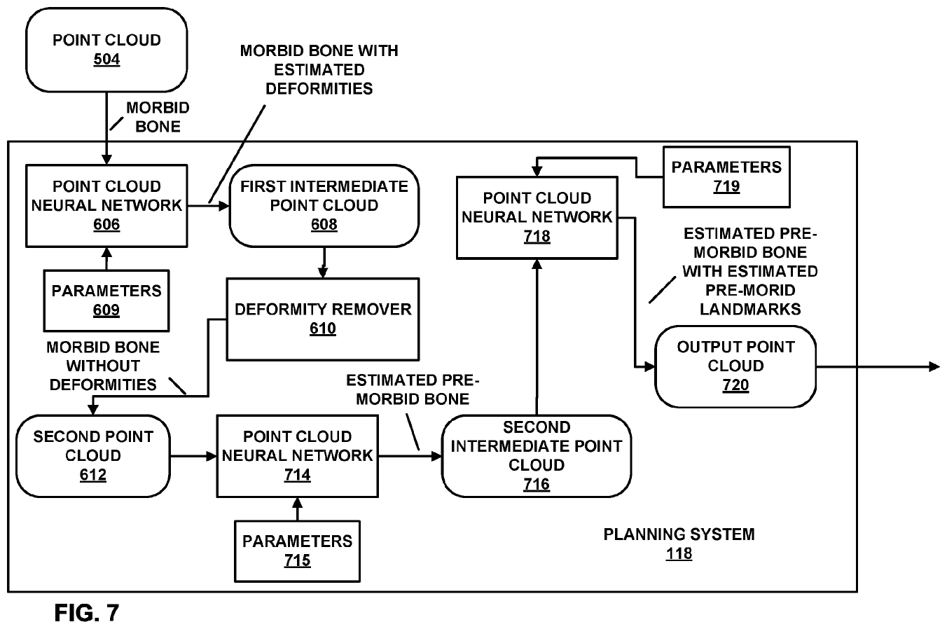

Point cloud network 506 of FIG. 5 directly generates output point cloud 508 that includes the estimated points of landmarks in a pre-morbid bone from an input point cloud (point cloud 504) that represents points on a morbid bone. Such a process is generally fast due to the use a single point cloud. In some circumstances, including for morbid bones exhibiting a great amount of disease or orientation, further accuracy of the location of pre-morbid landmarks may be achieved using two or more neural networks. Such additional examples are shown below with reference to FIG. 6 and FIG. 7.

FIG. 6 is a block diagram illustrating another example implementation of planning system according to another example of the disclosure. In the example of FIG. 6, planning system 118 again obtains and receives point cloud 504, which represents the morbid bone of a patient on which surgery is being planned. Point cloud 504 is processed by point cloud neural network 606. Different than point cloud neural network 506 of FIG. 5, point cloud neural network 606 outputs a first intermediate point cloud 608 which includes points of the morbid bone with estimated deformities. That is, the points of first intermediate point cloud 608 includes points labeled as being associated with a deformity. In this context, the deformities are additive deformities, such as ossifications.

As such, point cloud neural network 606 operates according to parameters 609 that were trained to identify deformities in a point cloud of a morbid bone, rather than just directly identifying pre-morbid landmarks. A training data set for point cloud neural network 606 may include a plurality of point clouds of morbid bones that include annotations indicating deformities. For example, one or more surgeons or other experts may annotate point clouds of morbid bones to indicate locations of deformities. Such training data sets may then be processed by point cloud neural network 606 and the predicted deformities in the output point cloud produced by point cloud neural network 606 may be compared to the annotated deformities (e.g., the ground truth points) and a loss function may be calculated. Based on the output of the loss function, the weights and bias values in parameters 609 may be updated and the training data set may be processed again to further refine the parameters. This process is iteratively repeated until a desired value for the loss function is achieved.

Deformity remover 610 may then remove any points identified as being deformities from first intermediate point cloud 608 to create second point cloud 612. Second point cloud 612 is then processed by point cloud neural network 614. Similar to point cloud neural network 506 of FIG. 5, point cloud neural network 614 is configured to generate output point cloud 616 that represents the morbid bone with estimated locations of pre-morbid landmarks. Point cloud neural network 614 operates according to parameters 615 that were trained to determine locations of landmarks on pre-morbid bones. However, different than point cloud neural network 506 of FIG. 5, point cloud neural network 614 may be trained with a training data set that is free of deformities. Because deformities may vary greatly from patient to patient, determining landmarks from input point clouds that include deformities may be difficult. By first removing the deformities, the consistency and accuracy of landmark estimation may be improved across a wider spectrum of potential input point clouds.

FIG. 7 is a block diagram illustrating another example implementation of planning system according to another example of the disclosure. In the example of FIG. 7, planning system again obtains and receives point cloud 504, which represents the morbid bone of a patient on which surgery is being planned. Point cloud 504 is processed by point cloud neural network 606. The same as in FIG. 6, point cloud neural network 606 outputs a first intermediate point cloud 608 which includes points of the morbid bone with estimated deformities. That is, first intermediate point cloud 608 includes points labeled as being associated with a deformity. In this context, the deformities are additive deformities, such as ossifications.

Deformity remover 610 may then remove any points identified as being deformities from first intermediate point cloud 608 to create second point cloud 612. Second point cloud 612 is then processed by point cloud neural network 714. Point cloud neural network 714 produces a second intermediate point cloud 716 that includes estimated points representing the shape of the patient's pre-morbid bone. In particular, point cloud neural network 714 may add points relative to second point cloud 612 to address any subtractive deterioration represented in the patient's pre-morbid bone. Accordingly, point cloud neural network 606 identifies additive deformities which are then removed by deformity remover 610. Then point cloud neural network 714 effectively “fills-in” points in areas of the morbid bone that have experienced subtractive deterioration to estimate a pre-morbid shape of the patient's bone. That is, second intermediate point cloud 716 includes more points than second point cloud 612 such that second intermediate point cloud 716 represents an estimate of the pre-morbid shape of the patient's bone.

Point cloud neural network 714 operates according to parameters 715 that were trained to determine estimated points of a pre-morbid bone. A training data set for point cloud neural network 714 may include a plurality of point clouds of morbid bones that include annotations indicating additional points representing estimated shape of a pre-morbid bones. For example, one or more surgeons or other experts may annotate point clouds of morbid bones to indicate points that may be present in a pre-morbid bone. Such training data sets may then be processed by point cloud neural network 714 and the predicted points in the output point cloud produced by point cloud neural network 714 may be compared to the annotated points (e.g., the ground truth points) and a loss function may be calculated. Based on the output of the loss function, the biases and weights in parameters 715 may be updated and the training data set may be processed again to further refine the parameters. This process is iteratively repeated until a desired value for the loss function is achieved.

Second intermediate point cloud 716 is then processed by point cloud neural network 718. Similar to point cloud neural network 506 of FIG. 5 and point cloud neural network 614 of FIG. 6, point cloud neural network 718 is configured to generate output point cloud 720 that represents the morbid bone with estimated locations of pre-morbid landmarks. Point cloud neural network 718 operates according to parameters 719 that were trained to determine locations of landmarks on pre-morbid bones. However, different than point cloud neural network 506 of FIG. 5 or point cloud neural network 614 of FIG. 6, point cloud neural network 718 may be trained with a training data set that is free of both additive and subtractive deformities. Because additive and subtractive deformities may vary greatly from patient to patient, determining landmarks from input point clouds that include such deformities may be difficult. By first removing the additive and subtractive deformities, the consistency and accuracy of landmark estimation may be improved across a wider spectrum of potential input point clouds.

FIG. 8 is a conceptual diagram illustrating an example technique for training a neural network in accordance with one or more techniques of this disclosure. FIG. 8 shows a general process that may be used for any point cloud neural network described in this disclosure. That is, point cloud neural network 800 may represent any of the neural networks in FIGS. 5-7 with the understanding that training data set 850 and ground truth points 854 may change based on the type of output desired from point cloud neural network 800. The example of FIG. 8 will be directed to training point cloud neural network 800 to estimate the location of pre-morbid landmarks in one or more bones.

Point cloud neural network 800 may be configured to process training data set 850 as an input. Training data set 850 may be a set of point clouds of a plurality of bones. Testing has shown that good training results can be received from 1000 different bone point clouds. However, more or fewer training point clouds (or 3D images) may be used based on the accuracy desired. Ground truth points 854 are the annotated version of training data set 850, where the points the training data set are labeled such that points associated with pre-morbid landmarks are identified.

Point cloud neural network 800 processes training data set 850 to produced predicted points 852. Predicted points 852 includes points that are predicted to locations of landmarks of pre-morbid bones. Loss function unit 860 may then compare each of the predicted points 852 with their corresponding ground truth points 854 to effectively determine the accuracy of the point cloud neural network 800. The output of the loss function is then used to determine updated parameters (e.g., weights of each output layer of point cloud neural network 800). These updated parameters replace the weights of parameters 809. The training process may be iteratively performed, and the parameters may be iteratively updated, over many instances of the training data set (e.g., called epochs) until a desired accuracy is achieved. That is, the training data set is fixed, but the same training date set may be entirely used again for each training iteration.

Each neural network training data set may include a plurality of data pairs, each data pair being made of the raw data plus expected labels. If the training data set includes fibula meshes, then the raw data is a fibula point cloud, the landmark to be estimated is the lateral malleolus, and the expected labels are the segmentation labels (is lateral malleolus/is not lateral malleolus) associated to each point of the raw point cloud.

For some example feedforward neural networks, the process of updating the parameters is called backpropagation. When training point cloud neural network 800, loss function unit 860 may compute a gradient of a loss function with respect to the weights (e.g., parameters 809) of point cloud neural network 800 for a single input-output example. Loss function unit 860 may perform a backpropagation algorithm that includes computing a gradient of the loss function with respect to each weight by a chain rule, computing the gradient one layer at a time, and iterating backward from the last layer to avoid redundant calculations of intermediate terms in the chain rule.

In general, loss function unit 860 performs backpropagation by computing a gradient in weight space of a feedforward neural network (e.g., point cloud neural network 800), with respect to a loss function. The input is a vector of features (e.g., a points or voxels of a 3D representation of a bone), and the target output is a segmentation of such features. For segmentation, the output may be a vector of probabilities that a particular point or voxel is or is not part of a landmark being estimated.

In one example, loss function unit 860 may use one or more of a plane loss, point loss, and weighed dice loss functions to train PCNN 800. For landmarks that are located on a plane or form planar areas of a bone, loss function unit 860 may calculate the following plane loss function (planeLoss):

$$planeLoss = - {\sum }_{pi ∈ P} {softmax}_{k} ( {P}_{i} ) < N , {N}_{{P}_{i}} > ,$$

where the points of the point cloud P forming a plane have the label k, Pi is the ith point of the point cloud, N_{Pi} is the unit vector normal to the bone surface at point Pi, and N is the unit normal of the plane being segmented. The function softmaxₖ is the output of the model of PCNN 800 and is a probability for a point to have the label k. A value close to one (1) indicates that the model of PCNN 800 has a high confidence that k is the right label for the point. The minus sign for the planeLoss function indicates that the training process attempts to minimize the value of the loss function.

Loss function unit 860 may also use a point loss function when it is desired to train PCNN 800 to find the location of a point landmark in a point cloud. All the points within a defined radius (r) from the landmark are given a label k. The model of PCNN 800 is trained to predict that points close to the desired landmark belong to the class k. In order to train the model of PCNN 800 using the proximity of the point landmark, loss function unit 860 may operate according to the following point loss function (pointLoss):

$$pointLoss = {\sum }_{pi ∈ P} {softmax}_{k} ( {P}_{i} ) {‖ l - {P}_{i} ‖}_{2} ,$$

where l is the point landmark to find.

Loss function unit 860 may also use a weighed dice loss since the classes may be unbalanced in some applications. For example, there may be fewer points belonging to remarkable regions (e.g., landmarks) of a bone than to the rest/background. The weights are generally inversely proportional to the number of points in each class. Thus, the classes that are less represented (e.g., landmarks to detect) have a bigger impact on the loss value so that these classes get more accurately segmented. The dice loss is a loss function extensively used for segmentation purposes.

In some examples, loss function unit 860 may use the plane loss, point loss, and weighted dice loss function and may combined the outputs to train the model of PCNN 800. Loss function unit 860 may be configured to linearly combine the above-described loss functions with weights that are customized according to the model/application. In other examples, loss function unit 860 may be configured to use more generic combinations using logarithmic, cosine, and/or polynomial expressions to accelerate and facilitate the minimization process and therefore obtain better results.

For segmentation, the last layer of point cloud neural network 800 is a logic faction that outputs a binary classification (e.g., is or is not part of a landmark). The training set for backpropagation includes set of input-output pairs. For each input-output pair in the training set, the loss of the model on that pair is the cost of the difference between the predicted output (e.g., predicted points 852) and the target output (ground truth points 854).

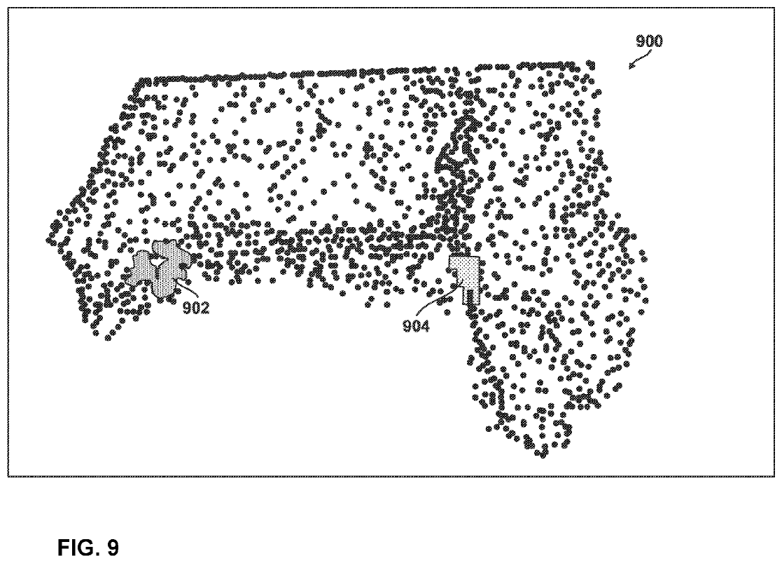

FIG. 9 is a conceptual diagram illustrating an example output point cloud 900 with labeled landmarks 902 and 904 in accordance with one or more techniques of this disclosure. Output point cloud 900 represents points located on the cortical of a distal tibia. Landmarks 902 show per-point labels corresponding to a medial gutter of the distal tibia. Landmarks 904 show per-point labels corresponding to a lateral gutter of the distal tibia.

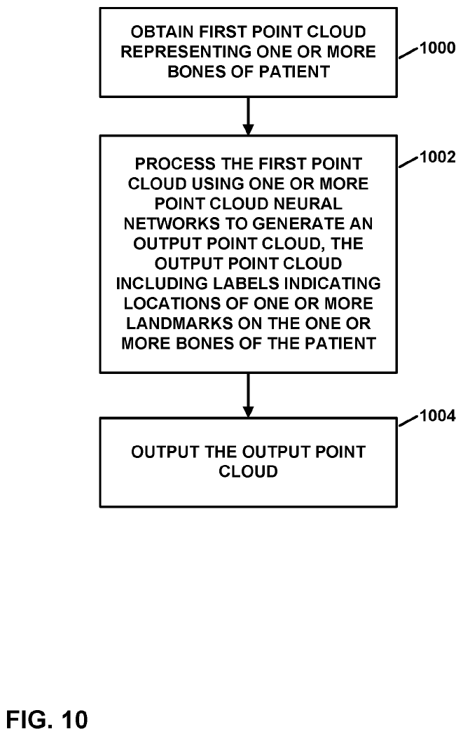

FIG. 10 is a flowchart illustrating an example process for estimating landmarks in accordance with one or more techniques of this disclosure. In the example of FIG. 10, computing system 102 may obtain a first point cloud representing one or more bones of a patient (1000). Computing system 102 may then process the first point cloud using one or more point cloud neural networks to generate an output point cloud that includes labels indicating locations of one or more landmarks on the one or more bones of the patient (1002). Computing system 102 may then output the output point cloud (1004).

In one example, the first point cloud represents one or more morbid bones of the patient. In one example, computing system 102 may process the first point cloud using a first point cloud neural network to generate the output point cloud. In this example, computing system 102 may be further configured to generate a training dataset based on point clouds of a plurality of morbid bones, and train the first point cloud neural network using the training dataset.

In another example, computing system 102 may process the first point cloud using a first point cloud neural network to generate a first intermediate point cloud, the first intermediate point cloud including labels indicating points representative of deformities on the one or more bones of the patient, and remove the points representative of the deformities from the first intermediate point cloud to generate a second point cloud. Computing system 102 may further process the second point cloud using a second point cloud neural network to generate the output point cloud, the output point cloud including labels indicating locations of one or more landmarks on the one or more bones of the patient.

In another example, computing system 102 may process the first point cloud using a first point cloud neural network to generate a first intermediate point cloud, the first intermediate point cloud including labels indicating points representative of deformities on the one or more bones of the patient, and remove the points representative of the deformities from the first intermediate point cloud to generate a second point cloud. In this example, computing system 102 may then process the second point cloud using a second point cloud neural network to generate a second intermediate point cloud, the second intermediate point cloud representing an estimation of a premorbid bone of the patient, and process the second intermediate point cloud using a third point cloud neural network to generate the output point cloud, the output point cloud including labels indicating locations of one or more landmarks on the one or more bones of the patient.

In any of the above examples, the output point cloud includes points representing a target bone of the patient and further includes labels indicating the locations of one or more landmarks on the target bone.

In any of the above examples, to process the first point cloud using one or more point cloud neural networks, computing system 102 may apply an input transform to a first array that comprises the first point cloud to generate a second array, wherein the input transform is implemented using a first T-Net model, apply a first multi-layer perceptron (MLP) to the second array to generate a third array, apply a feature transform to the third array to generate a fourth array, wherein the input transform is implemented using a second T-Net model, apply a second MLP to the fourth array to generate a fifth array, apply a max pooling layer to the fifth array to generate a global feature vector, sample N points in a unit square in 2-dimensions, concatenate the sampled points with the global feature vector to obtain a combined vector, and apply one or more third MLPs to generate points in the output point cloud.

In any of the above examples, computing system 102 may generate based on the output point cloud, a Mixed Reality visualization indicating the locations of one or more landmarks on the one or more bones of the patient.

In any of the above examples, the one or more bones include a tibia or a fibula, and wherein the landmarks include one or more of an anterior crest, a medial malleolus, a soleal line, a fibular facet, an articulation site for a head of the fibula, a fibular notch, an articulation site for a distal fibula, a point of ligamentous attachment, a proximal head of the fibula, a lateral malleolus, a facet on a distal end of the fibula, a medial condyle, a lateral condyle, a tibial plateau, a tibial tuberosity, or a site for articulation with a talus bone.

Other example aspects of the disclosure are described in the following example aspects.
Aspect 1—A method for estimating landmarks on a morbid bone, the method comprising: obtaining, by a computing system, a first point cloud representing one or more bones of a patient; processing, by the computing system, the first point cloud using one or more point cloud neural networks to generate an output point cloud, the output point cloud including labels indicating locations of one or more landmarks on the one or more bones of the patient; and outputting, by the computing system, the output point cloud.
Aspect 2—The method of Aspect 1, wherein the first point cloud represents one or more morbid bones of the patient, and wherein processing, by the computing system, the first point cloud using the one or more point cloud neural networks to generate the output point cloud comprises: processing, by the computing system, the first point cloud using a first point cloud neural network to generate the output point cloud.
Aspect 3—The method of Aspect 2, further comprising training the first point cloud neural network, wherein training the first point cloud neural network comprises: generating a training dataset based on point clouds of a plurality of morbid bones; and training the first point cloud neural network using the training dataset.
Aspect 4—The method of Aspect 1, wherein the first point cloud represents one or more morbid bones of the patient, and wherein processing, by the computing system, the first point cloud using the one or more point cloud neural networks to generate the output point cloud comprises: processing, by the computing system, the first point cloud using a first point cloud neural network to generate a first intermediate point cloud, the first intermediate point cloud including labels indicating points representative of deformities on the one or more bones of the patient; and removing the points representative of the deformities from the first intermediate point cloud to generate a second point cloud.
Aspect 5—The method of Aspect 4, further comprising: processing, by the computing system, the second point cloud using a second point cloud neural network to generate the output point cloud, the output point cloud including labels indicating locations of one or more landmarks on the one or more bones of the patient.
Aspect 6—The method of Aspect 4, further comprising: processing, by the computing system, the second point cloud using a second point cloud neural network to generate a second intermediate point cloud, the second intermediate point cloud representing an estimation of a premorbid bone of the patient; and processing, by the computing system, the second intermediate point cloud using a third point cloud neural network to generate the output point cloud, the output point cloud including labels indicating locations of one or more landmarks on the one or more bones of the patient.
Aspect 7—The method of any of Aspects 1-6, wherein the output point cloud includes points representing a target bone of the patient and further includes labels indicating the locations of one or more landmarks on the target bone.
Aspect 8—The method of any of Aspects 1-7, wherein processing, by the computing system, the first point cloud using one or more point cloud neural networks comprises: applying an input transform to a first array that comprises the first point cloud to generate a second array, wherein the input transform is implemented using a first T-Net model; applying a first multi-layer perceptron (MLP) to the second array to generate a third array; applying a feature transform to the third array to generate a fourth array, wherein the input transform is implemented using a second T-Net model; applying a second MLP to the fourth array to generate a fifth array; applying a max pooling layer to the fifth array to generate a global feature vector; sampling N points in a unit square in 2-dimensions; concatenating the sampled points with the global feature vector to obtain a combined vector; and applying one or more third MLPs to generate points in the output point cloud.
Aspect 9—The method of any of Aspects 1-8, further comprising: generating, by the computing system, based on the output point cloud, a Mixed Reality visualization indicating the locations of one or more landmarks on the one or more bones of the patient.
Aspect 10—The method of any of Aspects 1-9, wherein the one or more bones include a tibia or a fibula, and wherein the landmarks include one or more of an anterior crest, a medial malleolus, a soleal line, a fibular facet, an articulation site for a head of the fibula, a fibular notch, an articulation site for a distal fibula, a point of ligamentous attachment, a proximal head of the fibula, a lateral malleolus, a facet on a distal end of the fibula, a medial condyle, a lateral condyle, a tibial plateau, a tibial tuberosity, or a site for articulation with a talus bone.
Aspect 11—A computing system configured to estimate landmarks on a morbid bone, the computing system comprising: a memory configured to store a first point cloud representing one or more bones of a patient; and one or more processors in communication with the memory, the one or more processors configured to: obtain the first point cloud representing the one or more bones of the patient; process the first point cloud using one or more point cloud neural networks to generate an output point cloud, the output point cloud including labels indicating locations of one or more landmarks on the one or more bones of the patient; and output the output point cloud.
Aspect 12—The computing system of Aspect 11, wherein the first point cloud represents one or more morbid bones of the patient, and wherein to process the first point cloud using the one or more point cloud neural networks to generate the output point cloud, the one or more processors are further configured to: process the first point cloud using a first point cloud neural network to generate the output point cloud.
Aspect 13—The computing system of Aspect 12, wherein the one or more processors are further configured to train the first point cloud neural network, wherein to train the first point cloud neural network, the one or more processors are configured to: generate a training dataset based on point clouds of a plurality of morbid bones; and train the first point cloud neural network using the training dataset.
Aspect 14—The computing system of Aspect 11, wherein the first point cloud represents one or more morbid bones of the patient, and wherein to process the first point cloud using the one or more point cloud neural networks to generate the output point cloud, the one or more processors are configured to: process the first point cloud using a first point cloud neural network to generate a first intermediate point cloud, the first intermediate point cloud including labels indicating points representative of deformities on the one or more bones of the patient; and remove the points representative of the deformities from the first intermediate point cloud to generate a second point cloud.
Aspect 15—The computing system of Aspect 14, wherein the one or more processors are further configured to: process the second point cloud using a second point cloud neural network to generate the output point cloud, the output point cloud including labels indicating locations of one or more landmarks on the one or more bones of the patient.
Aspect 16—The computing system of Aspect 14, wherein the one or more processors are further configured to: process the second point cloud using a second point cloud neural network to generate a second intermediate point cloud, the second intermediate point cloud representing an estimation of a premorbid bone of the patient; and process the second intermediate point cloud using a third point cloud neural network to generate the output point cloud, the output point cloud including labels indicating locations of one or more landmarks on the one or more bones of the patient.
Aspect 17—The computing system of any of Aspects 11-16, wherein the output point cloud includes points representing a target bone of the patient and further includes labels indicating the locations of one or more landmarks on the target bone.
Aspect 18—The computing system of any of Aspects 11-17, wherein to process the first point cloud using one or more point cloud neural networks, the one or more processors are configured to: apply an input transform to a first array that comprises the first point cloud to generate a second array, wherein the input transform is implemented using a first T-Net model; apply a first multi-layer perceptron (MLP) to the second array to generate a third array; apply a feature transform to the third array to generate a fourth array, wherein the input transform is implemented using a second T-Net model; apply a second MLP to the fourth array to generate a fifth array; apply a max pooling layer to the fifth array to generate a global feature vector; sample N points in a unit square in 2-dimensions; concatenate the sampled points with the global feature vector to obtain a combined vector; and apply one or more third MLPs to generate points in the output point cloud.
Aspect 19—The computing system of any of Aspects 11-18, wherein the one or more processors are further configured to: generate based on the output point cloud, a Mixed Reality visualization indicating the locations of one or more landmarks on the one or more bones of the patient.
Aspect 20—The computing system of any of Aspects 11-19, wherein the one or more bones include a tibia or a fibula, and wherein the landmarks include one or more of an anterior crest, a medial malleolus, a soleal line, a fibular facet, an articulation site for a head of the fibula,
   a fibular notch, an articulation site for a distal fibula, a point of ligamentous attachment, a proximal head of the fibula, a lateral malleolus, a facet on a distal end of the fibula, a medial condyle, a lateral condyle, a tibial plateau, a tibial tuberosity, or a site for articulation with a talus bone.
Aspect 21—The computing system of any of Aspects 11-20, further comprising: a display configured to display the output point cloud.
Aspect 22—The computing system of Aspect 21, wherein the display is a visualization device.
Aspect 23—The computing system of Aspect 22, wherein the visualization device is one of a mixed reality (MR) visualization device, a virtual reality (VR) visualization device, a holographic projector, or an extended reality (XR) visualization device.
Aspect 24—A non-transitory computer-readable storage medium having instructions stored thereon that, when executed, cause a computing system to: obtain a first point cloud representing one or more bones of a patient; process the first point cloud using one or more point cloud neural networks to generate an output point cloud, the output point cloud including labels indicating locations of one or more landmarks on the one or more bones of the patient; and output the output point cloud.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuitry,” as used herein may refer to any of the foregoing structures or any other structure suitable for implementation of the techniques described herein.

1. A method for estimating landmarks on a morbid bone, the method comprising:
obtaining, by a computing system, a first point cloud representing one or more bones of a patient;
processing, by the computing system, the first point cloud using one or more point cloud neural networks to generate an output point cloud, the output point cloud including labels indicating locations of one or more landmarks on the one or more bones of the patient; and
outputting, by the computing system, the output point cloud.

2. The method of claim 1, wherein the first point cloud represents one or more morbid bones of the patient, and wherein processing, by the computing system, the first point cloud using the one or more point cloud neural networks to generate the output point cloud comprises:
processing, by the computing system, the first point cloud using a first point cloud neural network to generate the output point cloud.

3. The method of claim 2, further comprising training the first point cloud neural network, wherein training the first point cloud neural network comprises:
generating a training dataset based on point clouds of a plurality of morbid bones; and
training the first point cloud neural network using the training dataset.

4. The method of claim 1, wherein the first point cloud represents one or more morbid bones of the patient, and wherein processing, by the computing system, the first point cloud using the one or more point cloud neural networks to generate the output point cloud comprises:
processing, by the computing system, the first point cloud using a first point cloud neural network to generate a first intermediate point cloud, the first intermediate point cloud including labels indicating points representative of deformities on the one or more bones of the patient; and
removing the points representative of the deformities from the first intermediate point cloud to generate a second point cloud.

5. The method of claim 4, further comprising:
processing, by the computing system, the second point cloud using a second point cloud neural network to generate the output point cloud, the output point cloud including labels indicating locations of one or more landmarks on the one or more bones of the patient.

6. The method of claim 4, further comprising:
processing, by the computing system, the second point cloud using a second point cloud neural network to generate a second intermediate point cloud, the second intermediate point cloud representing an estimation of a premorbid bone of the patient; and
processing, by the computing system, the second intermediate point cloud using a third point cloud neural network to generate the output point cloud, the output point cloud including labels indicating locations of one or more landmarks on the one or more bones of the patient.

7. The method of claim 1, wherein the output point cloud includes points representing a target bone of the patient and further includes labels indicating the locations of one or more landmarks on the target bone.

8. The method of claim 1, wherein processing, by the computing system, the first point cloud using one or more point cloud neural networks comprises:
applying an input transform to a first array that comprises the first point cloud to generate a second array, wherein the input transform is implemented using a first T-Net model;
applying a first multi-layer perceptron (MLP) to the second array to generate a third array;
applying a feature transform to the third array to generate a fourth array, wherein the input transform is implemented using a second T-Net model;
applying a second MLP to the fourth array to generate a fifth array;
applying a max pooling layer to the fifth array to generate a global feature vector;
sampling N points in a unit square in 2-dimensions;
concatenating the sampled points with the global feature vector to obtain a combined vector; and
applying one or more third MLPs to generate points in the output point cloud.

9. The method of claim 1, further comprising:
generating, by the computing system, based on the output point cloud, a Mixed Reality visualization indicating the locations of one or more landmarks on the one or more bones of the patient.

10. (canceled)

11. The method of claim 1, further comprising:
determining one or more of a tibia mechanical axis, a tibia anatomical axis, a tibia axial plane, a tibia medial gutter line, a fibula lateral gutter line, a tibial mortise AP (anteroposterior) axis, or a tibial mortise ML (mediolateral) axis based on the locations of the one or more landmarks.

12. A computing system configured to estimate landmarks on a morbid bone, the computing system comprising:
a memory configured to store a first point cloud representing one or more bones of a patient; and
one or more processors in communication with the memory, the one or more processors configured to:
obtain the first point cloud representing the one or more bones of the patient;
process the first point cloud using one or more point cloud neural networks to generate an output point cloud, the output point cloud including labels indicating locations of one or more landmarks on the one or more bones of the patient; and
output the output point cloud.

13. The computing system of claim 12, wherein the first point cloud represents one or more morbid bones of the patient, and wherein to process the first point cloud using the one or more point cloud neural networks to generate the output point cloud, the one or more processors are further configured to:
process the first point cloud using a first point cloud neural network to generate the output point cloud.

14. The computing system of claim 13, wherein the one or more processors are further configured to train the first point cloud neural network, wherein to train the first point cloud neural network, the one or more processors are configured to:
generate a training dataset based on point clouds of a plurality of morbid bones; and
train the first point cloud neural network using the training dataset.

15. The computing system of claim 12, wherein the first point cloud represents one or more morbid bones of the patient, and wherein to process the first point cloud using the one or more point cloud neural networks to generate the output point cloud, the one or more processors are configured to:
process the first point cloud using a first point cloud neural network to generate a first intermediate point cloud, the first intermediate point cloud including labels indicating points representative of deformities on the one or more bones of the patient; and
remove the points representative of the deformities from the first intermediate point cloud to generate a second point cloud.

16. The computing system of claim 15, wherein the one or more processors are further configured to:
process the second point cloud using a second point cloud neural network to generate the output point cloud, the output point cloud including labels indicating locations of one or more landmarks on the one or more bones of the patient.

17. The computing system of claim 15, wherein the one or more processors are further configured to:
process the second point cloud using a second point cloud neural network to generate a second intermediate point cloud, the second intermediate point cloud representing an estimation of a premorbid bone of the patient; and
process the second intermediate point cloud using a third point cloud neural network to generate the output point cloud, the output point cloud including labels indicating locations of one or more landmarks on the one or more bones of the patient.

18. The computing system of claim 12, wherein the output point cloud includes points representing a target bone of the patient and further includes labels indicating the locations of one or more landmarks on the target bone.

19. The computing system of claim 12, wherein to process the first point cloud using one or more point cloud neural networks, the one or more processors are configured to:
apply an input transform to a first array that comprises the first point cloud to generate a second array, wherein the input transform is implemented using a first T-Net model;
apply a first multi-layer perceptron (MLP) to the second array to generate a third array;
apply a feature transform to the third array to generate a fourth array, wherein the input transform is implemented using a second T-Net model;
apply a second MLP to the fourth array to generate a fifth array;
apply a max pooling layer to the fifth array to generate a global feature vector;
sample N points in a unit square in 2-dimensions;
concatenate the sampled points with the global feature vector to obtain a combined vector; and
apply one or more third MLPs to generate points in the output point cloud.

20. The computing system of claim 12, wherein the one or more processors are further configured to:
generate based on the output point cloud, a Mixed Reality visualization indicating the locations of one or more landmarks on the one or more bones of the patient.

21. (canceled)

22. The computing system of claim 12, wherein the one or more processors are further configured to:
determine one or more of a tibia mechanical axis, a tibia anatomical axis, a tibia axial plane, a tibia medial gutter line, a fibula lateral gutter line, a tibial mortise AP (anteroposterior) axis, or a tibial mortise ML (mediolateral) axis based on the locations of the one or more landmarks.

23-26. (canceled)
